(19) United States
(12) Patent Application Publication
(10) Pub. No.: US 2011/0166228 A1
(43) Pub. Date: Jul. 7, 2011

(54) COMPOSITION

(76) Inventors: Anne Kristin Holmeide, Oslo (NO); Jenny Rosman, Marstrand (SE)

(21) Appl. No.: 12/446,249

(22) PCT Filed: Nov. 1, 2007
(86) PCT No.: PCT/IB07/04613

§ 371 Date: Mar. 19, 2011

Related U.S. Application Data
(60) Provisional application No. 60/855,733, filed on Nov. 1, 2006.
(60) Provisional application No. 60/856,267, filed on Nov. 3, 2006.
(60) Provisional application No. 60/856,268, filed on Nov. 3, 2006.
(60) Provisional application No. 60/856,269, filed on Nov. 3, 2006.

(30) Foreign Application Priority Data
Nov. 1, 2006 (SE) 0602310-5
Nov. 3, 2006 (SE) 0602352-7

(51) Int. Cl. A61K 31/202; A61P 3/10; A61P 9/10

(52) U.S. Cl. 514/560

(57) ABSTRACT

The present invention relates to compositions comprising at least omega-3 lipid compounds substituted at the 2-positions having therapeutic activity. More specifically, the present invention relates to a composition comprising at least omega-3 lipid compounds substituted at the 2-position, counted from the functional group (X) of the omega-3 lipid compound, wherein the omega-3 lipid compounds comprise: a compound of general formula (I):

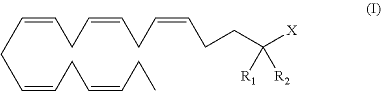

and a compound of formula (II):

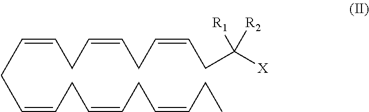

wherein R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; and X represents a carboxylic acid or a derivative thereof, a carboxylate, a carboxylic anhydride, a hydroxymethyl (—CH₂OH) or a pro-drug thereof, or a carboxamide, or any pharmaceutically acceptable complex, salt, solvate, or pro-drug, with the provisos that: R₁ and R₂ are not simultaneously hydrogen.

### FIELD OF THE INVENTION

The present invention relates to compositions comprising at least omega-3 lipid compounds substituted at the 2-position having therapeutic activity. More specifically, the present invention relates to a composition comprising at least omega-3 lipid compounds substituted at the 2-position, counted from the functional group (X) of the omega-3 lipid compound,

wherein the omega-3 lipid compounds comprise:

a compound of general formula (I):

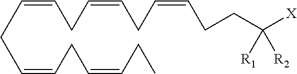

and a compound of formula (II):

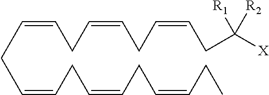

wherein
R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; and
X represents a carboxylic acid or a derivative thereof, a carboxylate, a carboxylic anhydride, a hydroxymethyl (—CH₂OH) or a pro-drug thereof, or a carboxamide,
   or any pharmaceutically acceptable complex, salt, solvate, or pro-drug thereof,

with the provisos that:

R₁ and R₂ are not simultaneously hydrogen.

Non-limiting examples of pro-drugs of the present invention include pivaloate esters, hemisuccinate esters or salts thereof.

The invention also relates to a composition comprising the salts of the compounds of formula (I) and (II). Such salts may be represented by

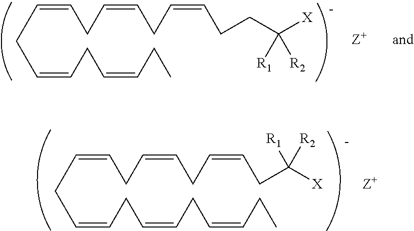

wherein X is COO⁻,
Z⁺ is selected from the group consisting of Li⁺, Na⁺, K⁺, NH₄⁺,

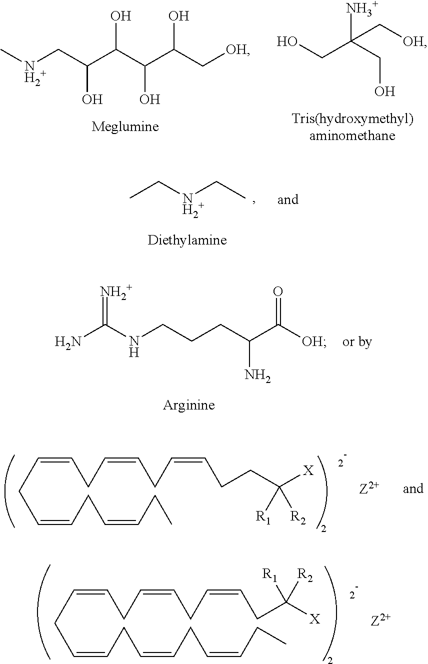

wherein X═COO⁻,
Z²⁺ is selected from the group consisting of Mg²⁺, Ca²⁺,

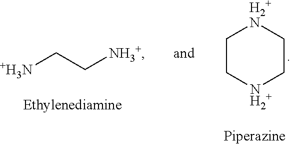

Another representative salt is

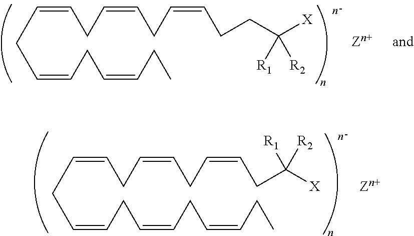

wherein X is COO⁻,

### Zⁿ⁺ is

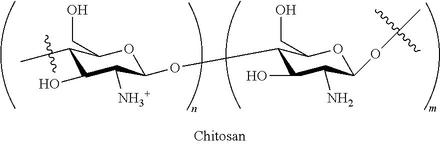

Furthermore, the present invention relates to compositions comprising compounds of formula (I) and (II), wherein X is a carboxylic acid in the form of a phospholipid. Such compounds may be represented by the following formulas,

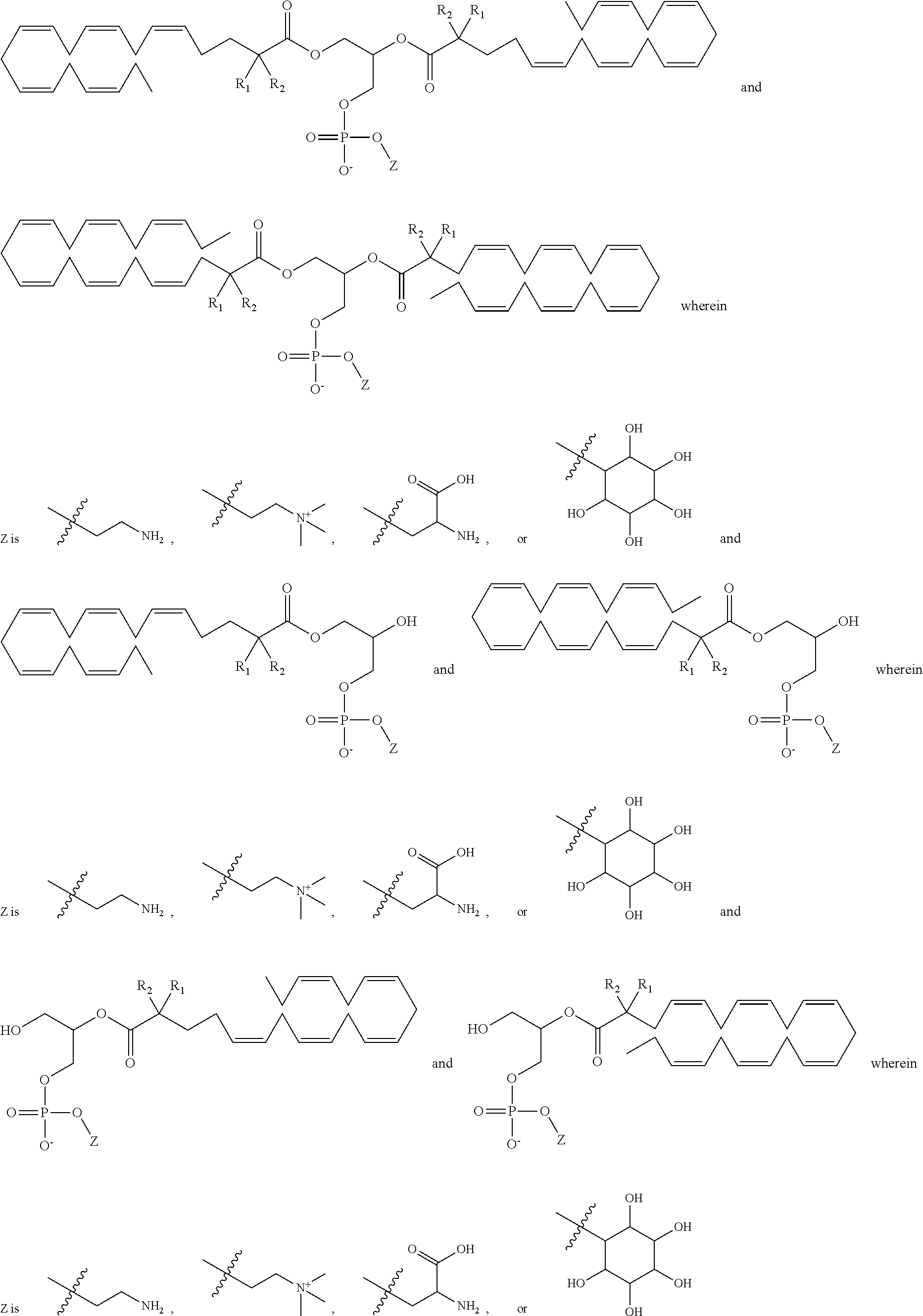

Compounds of formulas (I) and (II), wherein X is a carboxylic acid in the form of a triglyceride, a 1-monoglyceride and a 2-monoglyceride are also included in the present invention. These are hereinafter represented by the following formulas, respectively.

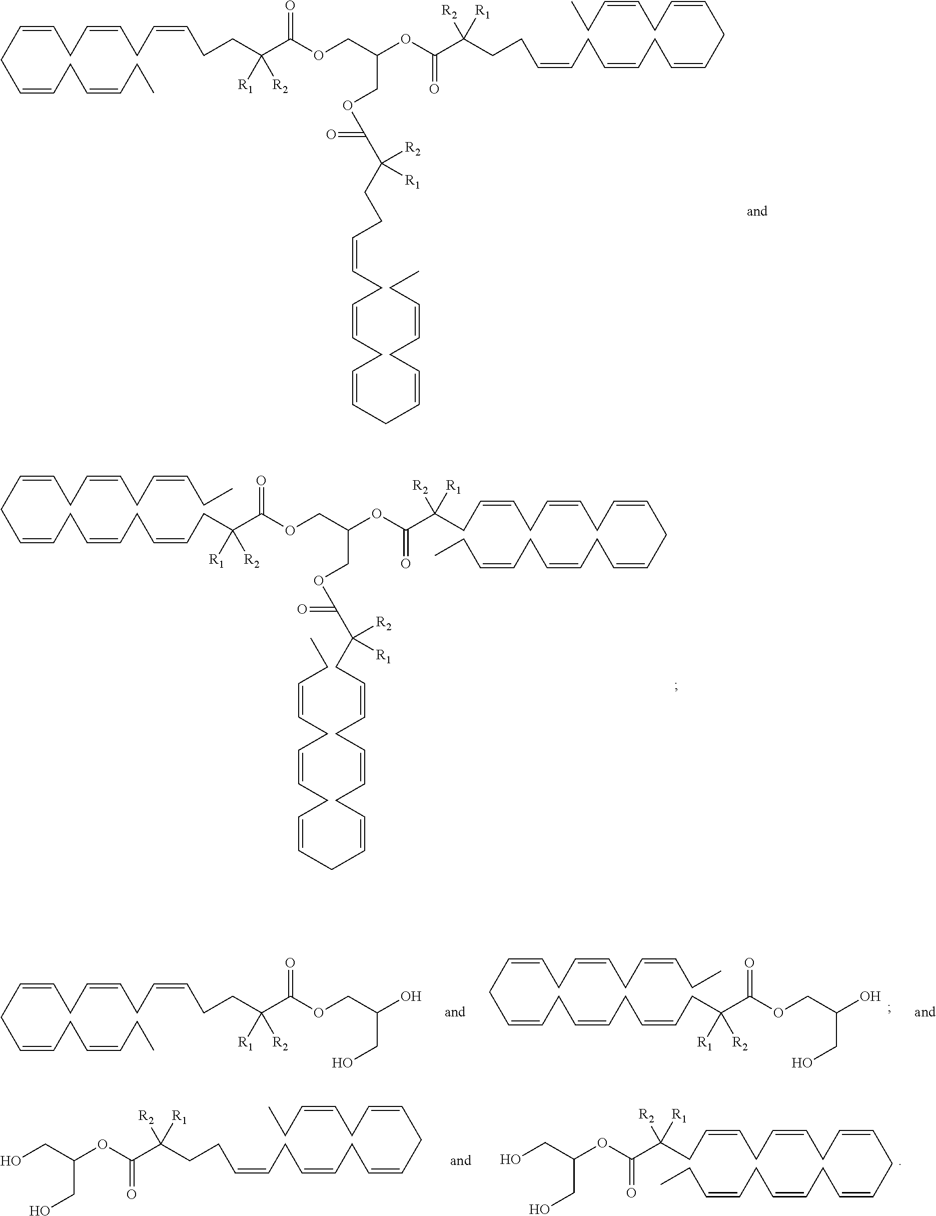

The present invention also relates to the use of the composition for the production of medicaments, as well as methods for treatment using the composition according to the invention. Finally the invention also relates to a method for the preparation of the above composition.

### BACKGROUND OF THE INVENTION

Dietary polyunsaturated fatty acids (PUFAs) have effects on diverse physiological processes impacting normal health and chronic diseases, such as the regulation of plasma lipid levels, cardiovascular and immune functions, insulin action, and neuronal development and visual function. Ingestion of PUFAs (generally in ester form, e.g. in glycerides or phospholipids) will lead to their distribution to virtually every cell in the body effecting the membrane composition and function, eicosanoid synthesis, cellular signalling and regulation of gene expression. Variations in distribution of different fatty acids/lipids to different tissues in addition to cell specific lipid metabolism, as well as the expression of fatty acid-regulated transcription factors, is likely to play an important role in determining how cells respond to changes in PUFA composition. (Benatti, P. Et al, J. Am. Coll. Nutr. 2004, 23, 281). PUFAs or their metabolites have been shown to modulate gene transcription by interacting with several nuclear receptors. These are the peroxisome proliferators-activated receptors (PPARs), the hepatic nuclear receptor (HNF-4), liver X receptor (LXR), and the 9-cis retinoic acid receptor (retinoic X receptor, RXR). Treatment with PUFAs can also regulate the abundance of many transcriptional factors in the nucleus, including SREBP, NFkB, c/EBPβ, and HIF-1α. These effects are not due to direct binding of the fatty acid to the transcription factor, but involve mechanisms that affect the nuclear content of the transcription factors. The regulation of gene transcription by PUFAs have profound effects on cell and tissue metabolism and offer a credible explanation for the involvement of nutrient-gene interactions in the initiation and prevention or amelioration of diseases such as obesity, diabetes, cardiovascular disorders, immune-inflammatory diseases and cancers (Wahle, J., et al, Proceedings of the Nutrition Society, 2003, 349). Fish oils rich in the omega-3 polyunsaturated fatty acids eicosapentaenoic acid (EPA) and docosahexaenoic acid (DHA), have been shown to reduce the risk of cardiovascular diseases partly by reduction of blood triglyceride concentration. This favorable effect mainly results from the combined effects of inhibition of lipogenesis by decrease of SPEBP-1 and stimulation of fatty acid oxidation by activation of PPAR-α in the liver.

ω-3 polyunsaturated fatty acids in fish oil have been reported to improve the prognosis of several chronic inflammatory diseases characterized by leukocyte accumulation and leukocyte-mediated tissue injury, including atherosclerosis, IgA nephropathy, inflammatory bowel disease, rheumatoid arthritis, psoriasis, etc. (Mishra, A., Arterioscler. Thromb. Vasc. Biol., 2004, 1621).

Due to their limited stability in vivo and their lack of biological specificity, PUFAs have not achieved widespread use as therapeutic agents. Chemical modifications of the n-3 polyunsaturated fatty acids have been performed by several research groups in order to change or increase their metabolic effects.

For example, the hypolipidemic effects of EPA was potentiated by introducing methyl or ethyl in α-position of EPA EE. (Vaagenes 1999). The compound also reduced plasma free fatty acids while EPA EE had no effect.

In a recent work published by L. Larsen (Larsen, L. et al, Lipids, 2005, 40, 49) the authors show that the α-methyl derivatives of EPA and DHA increased the activation of the nuclear receptor PPARα and thereby the expression of L-FABP compared to EPA/DHA. The authors suggest that delayed catabolism of these α-methyl PUFAs contribute to their increased effects.

Nuclear receptors (NRs) constitute a large and highly conserved family of ligand activated transcriptional factors that regulate diverse biological processes such as development, metabolism, and reproduction. It is recognized that ligands for these receptors might be used in the treatment of common diseases such as atherosclerosis, diabetes, obesity, and inflammatory diseases. As such, NRs have become important drug targets, and the identification of novel NR ligands is a subject of much interest. The activity of many nuclear receptors is controlled by the binding of small, lipophilic ligands that include hormones, metabolites such as fatty acids, bile acids, oxysteroles and xeno- and endobiotics. Nuclear receptors can bind as monomers, homodimers, or RXR heterodimers to DNA.

The transcription factor NF-κB is an inducible eukaryotic transcription factor of the rel family. It is a major component of the stress cascade that regulates the activation of early response genes involved in the expression of inflammatory cytokines, adhesion molecules, heat-shock proteins, cyclooxygenases, lipoxygenases, and redox enzymes. Zhao, G. et al (Biochemical and Biophysical Research Comm., 2005, 909) suggest that the anti-inflammatory effects of PUFAs in human monocytic THP-1 cells are in part mediated by inhibition of NF-κB activation via PPAR-γ activation. Others have suggested that the anti-inflammatory effect of PUFAs is mediated through a PPAR-α dependent inhibition of NF-κB activation.

### SUMMARY OF THE INVENTION

The aim of the present invention is to provide new compositions having therapeutic activity. This object is achieved by a composition comprising at least omega-3 lipid compounds substituted at the 2-position, wherein the omega-3 lipid compounds comprise:

a compound of formula (I):

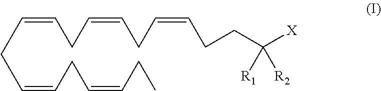

and a compound of formula (II):

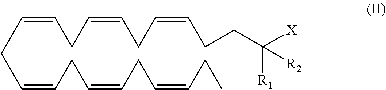

wherein R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; and
X represents a carboxylic acid or a derivative thereof, a carboxylate, a carboxylic anhydride, a hydroxymethyl (—CH₂OH) group or a pro-drug thereof, or a carboxamide,
with the proviso that R₁ and R₂ are not simultaneously a hydrogen atom.

A preferred embodiment includes a composition comprising at least α-ethyl EPA in the form of a tri-glyceride and α-ethyl DHA in the form of a tri-glyceride.

In particular, the present invention relates to a composition comprising at least omega-3 lipid compounds substituted at the 2-position, wherein the omega-3 lipid compounds comprise:

a compound of formula (I):

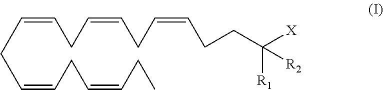

and a compound of formula (II):

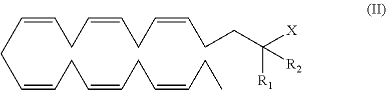

wherein
the weight ratio of compounds of formula (I):compounds of formula (II) is from 1:10 to 10:1, and

wherein
R₁ and R₂ are the same or different and are chosen from the methyl, ethyl, propyl, dimethyl, diethyl, thiomethyl, thioethyl, methoxy, ethoxy, hydroxy, methylamino and ethylamino; and
X represents a carboxylic acid or a derivative thereof, a carboxylate, a carboxylic anhydride, a hydroxymethyl (—CH₂OH), or a carboxamide.

Moreover, the present invention also relates to a composition comprising at least omega-3 lipid compounds substituted at the 2-position, wherein the omega-3 lipid compounds comprise:

a compound of formula (I):

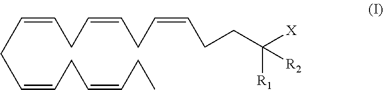

and a compound of formula (II):

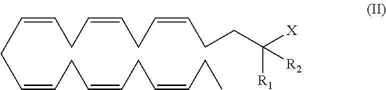

wherein
R₁ and R₂ are the same or different and are chosen from methyl, ethyl, propyl, dimethyl, diethyl, thiomethyl, thioethyl, methoxy, ethoxy, hydroxy, methylamino and ethylamino; and
X represents a hydroxymethyl (—CH₂OH).

In the compounds of formulas (I) and (II), X typically represents and ethylcarboxylate or a carboxylic acid. However, X may also be a derivative of a carboxylic acid in the form of a phospholipid or a tri-, di-, or monoglyceride.

In a composition according to the invention, said alkyl group may be chosen from methyl, ethyl, n-propyl, isopropyl, n-butyl, sec-butyl, n-hexyl, and n-heptyl; said halogen atom may be chosen from fluorine, chlorine, bromine, and iodine; said alkoxy group may be chosen from methoxy, ethoxy, propoxy, isopropoxy, sec-butoxy, phenoxy, benzyloxy, OCH₂CF₃, and OCH₂CH₂OCH₃; said acyloxy group may be chosen from acetoxy, propionoxy, and butyroxy; said alkenyl group may be chosen from allyl, 2-butenyl, and 3-hexenyl; said alkynyl group may be chosen from propargyl, 2-butynyl, and 3-hexynyl; said aryl group may be chosen from benzyl and a substituted benzyl group; said alkylthio group may be chosen from methylthio, ethylthio, isopropylthio, and phenylthio; said alkoxycarbonyl group may be chosen from methoxycarbonyl, ethoxycarbonyl, propoxycarbonyl, and butoxycarbonyl; said alkylsulfinyl group may be chosen from methanesulfinyl, ethanesulfinyl, and isopropanesulfinyl; said alkylsulfonyl group may be chosen from methanesulfonyl, ethanesulfonyl, and isopropanesulfonyl; said alkylamino group may be chosen from methylamino, dimethylamino, ethylamino, and diethylamino; said carboxylate group may be chosen from ethyl carboxylate, methyl carboxylate, n-propyl carboxylate, isopropyl carboxylate, n-butyl carboxylate, sec-butyl carboxylate, and n-hexyl carboxylate; said carboxamide group may be chosen from primary carboxamide, N-methyl carboxamide, N,N-dimethyl carboxamide, N-ethyl carboxamide, and N,N-diethyl carboxamide.

In an exemplary embodiment of the invention, R₁ and R₂ are chosen from a hydrogen atom, a hydroxy group, an alkyl group, a halogen atom, an alkoxy group, an alkylthio group, an alkylsulfinyl group, an alkylsulfonyl group, an amino group, and an alkylamino group.

In another embodiment of the invention, R₁ and R₂ are chosen from a hydrogen atom, a hydroxy group, a C₁-C₇ alkyl group, a halogen atom, a C₁-C₇ alkoxy group, a C₁-C₇ alkylthio group, a C₁-C₇ alkylsulfinyl group, a C₁-C₇ alkylsulfonyl group, an amino group, and a C₁-C₇ alkylamino group. Then, said C₁-C₇ alkyl group may be methyl, ethyl, or benzyl; said halogen atom may be fluorine: said C₁-C₇ alkoxy group may be methoxy or ethoxy; said C₁-C₇ alkylthio group may be methylthio, ethylthio or phenylthio; said C₁-C₇ alkylsulfinyl group may be ethanesulfinyl; said C₁-C₇ alkylsulfonyl group may be ethanesulfonyl; said C₁-C₇ alkylamino group may be ethylamino or diethylamino; and X may represent an ethyl carboxylate or a carboxamide group.

In another embodiment of the invention, R₁ and R₂ are chosen from a hydrogen atom, a C₁-C₃ alkyl group, and a C₁-C₃ alkoxy group, and X represents a carboxylate or a hydroxymethyl (—CH₂OH).

Examples of compositions, comprising omega-3 lipid compounds of formula (I) and (II), according to the invention are those in which X is a ethylcarboxylate, and wherein;
one of R₁ and R₂ is methyl and the other one is a hydrogen atom;
one of R₁ and R₂ is ethyl and the other one is a hydrogen atom; P1 one of R₁ and R₂ is propyl and the other one is a hydrogen atom; P1 one of R₁ and R₂ is methoxy and the other one is a hydrogen atom; P1 one of R₁ and R₂ is ethoxy and the other one is a hydrogen atom; P1 one of R₁ and R₂ is propoxy and the other one is a hydrogen atom; P1 one of R₁ and R₂ is thiomethyl and the other one is a hydrogen atom; P1 one of R₁ and R₂ is thioethyl and the other one is a hydrogen atom; P1 one of R₁ and R₂ is thiopropyl and the other one is a hydrogen atom;
one of R₁ and R₂ is ethylamino and the other one is a hydrogen atom; P1 one of R₁ and R₂ is diethylamino and the other one is a hydrogen atom; or P1 one of R₁ and R₂ is amino and the other one is a hydrogen atom.

Other examples of compositions, comprising omega-3 lipid compounds of formula (I) and (II), according to the invention are those in which X is a hydroxymethyl and wherein;
one of R₁ and R₂ is methyl and the other one is a hydrogen atom; P1 one of R₁ and R₂ is ethyl and the other one is a hydrogen atom; P1 one of R₁ and R₂ is propyl and the other one is a hydrogen atom; P1 one of R₁ and R₂ is methoxy and the other one is a hydrogen atom; P1 one of R₁ and R₂ is ethoxy and the other one is a hydrogen atom; P1 one of R₁ and R₂ is propoxy and the other one is a hydrogen atom;
one of R₁ and R₂ is thiomethyl and the other one is a hydrogen atom; P1 one of R₁ and R₂ is thioethyl and the other one is a hydrogen atom; P1 one of R₁ and R₂ is thiopropyl and the other one is a hydrogen atom; P1 one of R₁ and R₂ is ethylamino and the other one is a hydrogen atom;
one of R₁ and R₂ is diethylamino and the other one is a hydrogen atom; or P1 one of R₁ and R₂ is amino and the other one is a hydrogen atom.

In the compounds according to formula (I) and formula (II) of the present invention, R₁ and R₂ may be the same or different. When they are different, the compounds of formula (I) and (II) are capable of existing in stereoisomeric forms. It will be understood that the invention encompasses all optical isomers of the compounds of formula (I) and (II) and mixtures thereof including racemates. Therefore, the present invention includes, where R₁ is different from R₂, compositions comprising compounds of formula (I) and of formula (II) that are racemic or enantiomerically pure, either as the (S) or (R) enantiomer. Therefore, the present invention includes, where R₁ is different from R₂, compositions comprising compounds of formula (I) and of formula (II) that are racemic or enantiomeric pure, either as the (S) or (R) stereoisomer.

Another aspect of the present invention relates to a composition comprising at least one compound of formula (I) and a compound of formula (II) for use as a medicament.

Further, the present invention relates to a pharmaceutical composition comprising omega-3 lipid compounds according to the invention. The pharmaceutical composition may comprise a pharmaceutically acceptable carrier, excipient or diluent, or any combination thereof, and is suitably formulated for oral administration, e.g. in the form of a capsule or a sachet. A suitable daily dosage of the compound according to formula (I) is 5 mg to 10 g of said compound; 50 mg to 1 g of said compound, or 50 mg to 200 mg of said compound. A suitable daily dosage of the of the compound according to formula (II) is 5 mg to 10 g of said compound; 50 mg to 1 g of said compound, or 50 mg to 200 mg of said compound. A suitable daily dosage of the composition is 5 mg to 10 g; 50 mg to 1 g of said compound; or 50 mg to 200 mg.

Further, the invention relates to the use of omega-3 lipid compounds according to the invention for the production of a medicament for:
activation or modulation of at least one of the human peroxisome proliferator-activated receptor (PPAR) isoforms, wherein said peroxisome proliferator-activated receptor (PPAR) is peroxisome proliferator-activated receptor (PPAR)α and/or γ;
the treatment and/or the prevention of peripheral insulin resistance and/or a diabetic condition;
reduction of plasma insulin, blood glucose and/or serum triglycerides;
the treatment and/or the prevention of type 2 diabetes;
the prevention and/or treatment of elevated triglyceride levels, LDL cholesterol levels, and/or VLDL cholesterol levels;
the prevention and/or treatment of a hyperlipidemic condition, e.g. hypertriglyceridemia (HTG);
increasing serum HDL levels in humans;
the treatment and/or the prevention of obesity or an overweight condition;
reduction of body weight and/or for preventing body weight gain;
the treatment and/or the prevention of a fatty liver disease, e.g. non-alcoholic fatty liver disease (NAFLD);
treatment of insulin resistance, hyperlipidemia and/or obesity or an overweight condition; and
the production of a medicament for the treatment and/or the prevention of an inflammatory disease or condition.

The invention also relates to methods for the treatment and/or prevention of the conditions listed above, comprising administering to a mammal in need thereof a pharmaceutically active amount of a composition comprising omega-3 lipid compounds substituted at the 2-position,

wherein the omega-3 lipid compounds comprise:

a compound of formula (I):

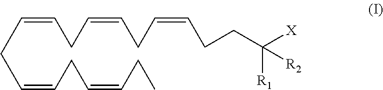

and a compound of formula (II):

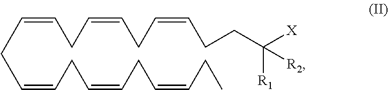

wherein R₁ and R₂ are hereinabove defined.

In addition, the present invention encompasses a process for the manufacture of a composition comprising omega-3 lipid compounds substituted at the 2-position, wherein the omega-3 lipid compounds comprise:

a compound of formula (I):

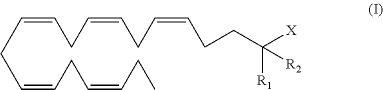

and a compound of formula (II):

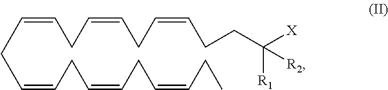

wherein R₁ and R₂ are hereinabove defined.

The raw material may e.g. originate from a vegetable, a microbial and/or an animal source, such as a marine fish oil. Preferably a marine oil or a krill oil is used.

### DESCRIPTION OF THE EMBODIMENTS

It has been shown that α-substituted derivatives of EPA and α-substituted derivatives of DHA have higher affinity to nuclear receptors of the PPAR family. To produce these derivatives, purified EPA or DHA have to be obtained as starting material. This process is quite complex and the yields are often low. The present invention describes α-substituted compositions derived from omega-3 concentrates. Because natural oils rich in omega-3 polyunsaturated fatty acids contain both eicosapentaenoic acid and docosapentaenoic acid in addition to other polyunsaturated fatty acids it could be a great benefit to produce α-substituted polyunsaturated fatty acids derivatives directly from omega-3 compositions. The present invention therefore relates to such polyunsaturated lipid/fatty acid compositions substituted in α-position and their use in therapy.

In addition to being better ligands for nuclear receptors the derivatives of the invention are not as easily degraded by α- and β-oxidation pathways as natural PUFAs due to substitution in α-position.

Research within the field of omega-3 fatty acid over the last several years has identified mechanisms underlying their biological effects. It is apparent that the physiological activity of the different omega-3 polyunsaturated fatty acids depends on their structure. It seems that structural elements, such as chain length and number of double bonds, have an impact on their effects. Animal studies have shown that DHA and EPA accumulate in different compartments in the body and may be metabolized differently.

Differences in the accumulation and retention of DHA and EPA may be related to the lipid moieties in which these fatty acids are stored or transported. DHA is incorporated predominantly in phospholipids, with a lesser portion accumulating in triacylglycerol and sterol esters, whereas EPA is more equally distributed between neutral lipids (sterol esters and triacylglycerol) and phospholipids.

Because the nuclear receptors are expressed in different tissues it is beneficial to target the tissues where the desired nuclear receptors are expressed with the agonist/modulator. A mixture of PPAR agonists derived from EPA and DHA will be more widely distributed than each of those derivatives alone. Consequently, the therapeutic effects on selected diseases should increase.

The alcohols and anhydrides of the compounds/compositions covered by the invention can be covered by this prodrug definition. A prodrug is defined as: Any compound that undergoes a biotransformation before exhibiting its pharmacological effects. Prodrugs can thus be viewed as drugs containing specialized non-toxic protective groups used in a transient manner to alter or to eliminate undesirable properties in the parent molecule. The compounds covered by the invention wherein X is a hydroxymethyl might be in the form of a prodrug of the alcohol, i.e. an acetate, hemisuccinate, phosphonate, sulphonate, or pivaloate ester.

Nomenclature and Terminology

Fatty acids are straight chain hydrocarbons possessing a carboxyl (COOH) group at one end (α) and (usually) a methyl group at the other (ω) end. In physiology, fatty acids are named by the position of the first double bond from the w end. The term ω-3 (omega-3) signifies that the first double bond begins at the third carbon-carbon bond from the terminal CH₃ end (ω) of the carbon chain. In chemistry, the numbering of the carbon atoms starts from the α end.

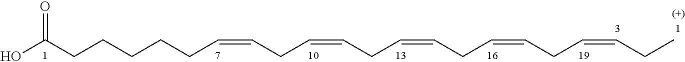

Throughout this specification, the terms “2-substituted”, “substituted in position 2”, and “substituted at carbon 2, counted from the functional group (X) of the omega-3 lipid compound” refers to substitution at the carbon atom denoted 2 in accordance with the above numbering of the carbon chain. Alternatively, such a substitution may be called an “alpha substitution”.

Throughout this specification, the term “omega-3 lipid compound” (corresponding to ω-3, or n-3) relates to a lipid compound having the first double bond at the third carbon-carbon bond from the ω end of the carbon chain, as defined above.

The basic idea of the present invention is a composition comprising omega-3 lipid compounds substituted at the 2-position, wherein the omega-3 lipid compounds comprise:

a compound of formula (I):

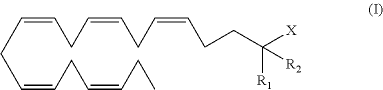

and a compound of formula (II):

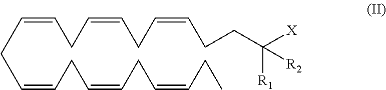

wherein R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; and
X represents a carboxylic acid or a derivative thereof, a carboxylate, a carboxylic anhydride, a hydroxymethyl (—CH₂OH) group or a pro-drug thereof, or a carboxamide,
with the proviso that R₁ and R₂ are not simultaneously a hydrogen atom.

The resulting compounds are alpha substituted omega-3 lipid compounds, i.e. an omega-3 lipid compound substituted in position 2 of the carbon atom, counted from the carbonyl end. More particularly, the resulting compounds are alpha substituted polyunsaturated lipids, which may be present as a carboxylic acid, or a derivative thereof, as a hydroxymethyl, as a carboxylate, as a carboxylic anhydride or as a carboxamide.

A preferred composition according to the invention includes omega-3 lipid compounds substituted at the 2-position, in a concentration of at least 30% by weight of the total lipid content of the composition, preferably at least 50% by weight, more preferably at least 60% by weight, still more preferably at least 70% by weight, and most preferably at least 80% by weight.

In an exemplary embodiment of the invention, the compounds of formulas (I) and (II) are present in a concentration of at least 20% by weight, more preferably at least about 40% by weight, still more preferably at least about 70% by weight, and most preferably at least about 80% by weight, of all of the omega-3 lipid compounds substituted at the 2-position.

Preferably the compound of formula (I) is present in a concentration of about 5% to about 95% by weight, preferably about 40% to about 55% by weight, of the total lipid content in the composition.

Preferably the compound of formula (II) is present in a concentration of about 5% to about 95% by weight, preferably about 30% to about 60% by weight, of the total lipid content in the composition.

In still another embodiments of the invention, the weight ratio of compounds of formula (I):compounds of formula (II) in the composition is from 1:99 to 99:1, more preferably from 10:1 to 1:10, still more preferably from 1:5 to 5:1, most preferably from 1:3 to 3:1.

In an exemplary embodiment, the weight ratio of compounds of formula (I):compounds of formula (II) in the composition is from 1:2 to 2:1, wherein at least one of R₁ and R₂ is ethyl, and X is an ethyl carboxylate or a hydroxymethyl.

It is to be understood that the present invention encompasses any possible pharmaceutically acceptable complexes, solvates or pro-drugs of the omega-3 lipid compounds of formula (I) and (II).

In a specific embodiment of the invention, the composition comprising at least omega-3 lipid compounds substituted at carbon 2, counted from the functional group of the omega-3 lipid compound, which omega-3 lipid compounds comprising at least:

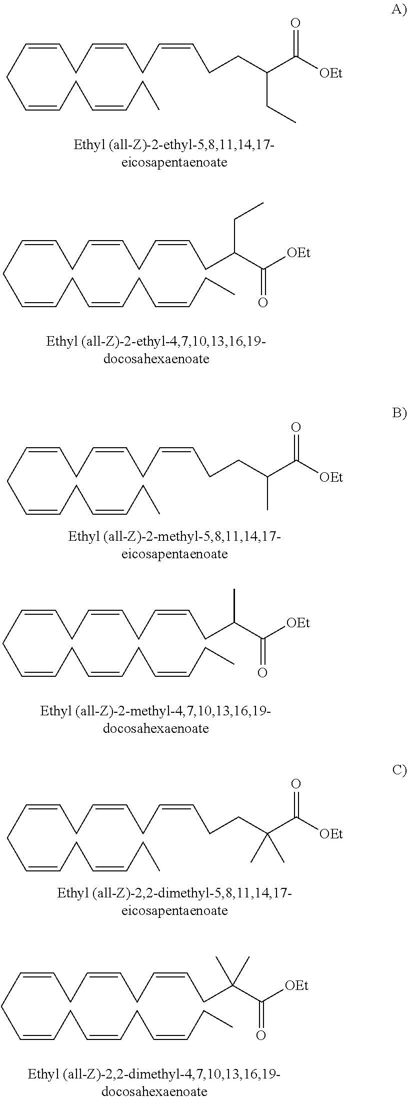

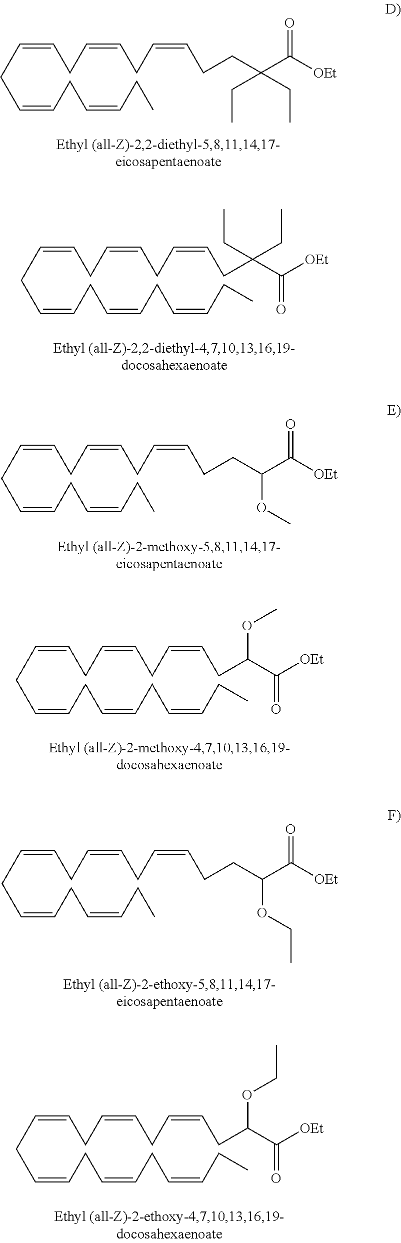

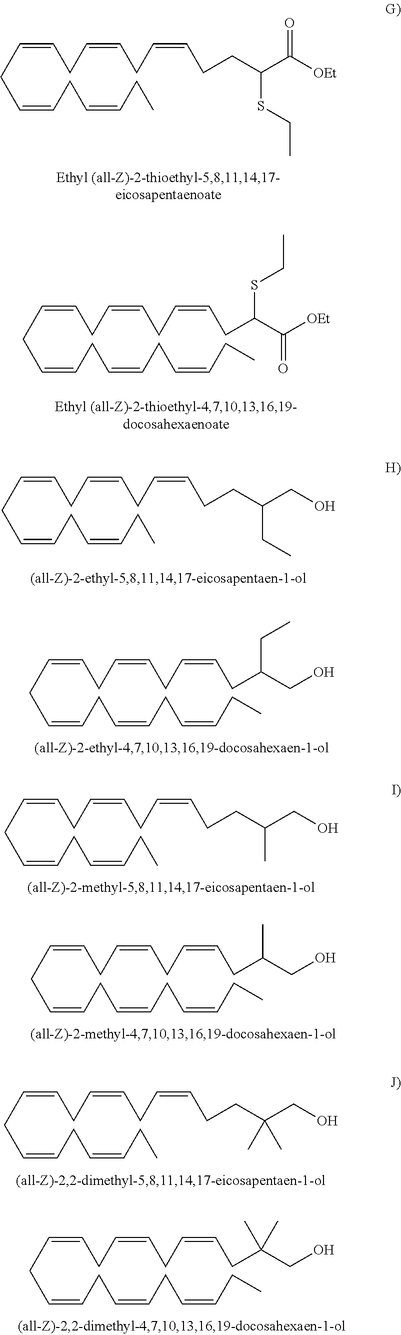

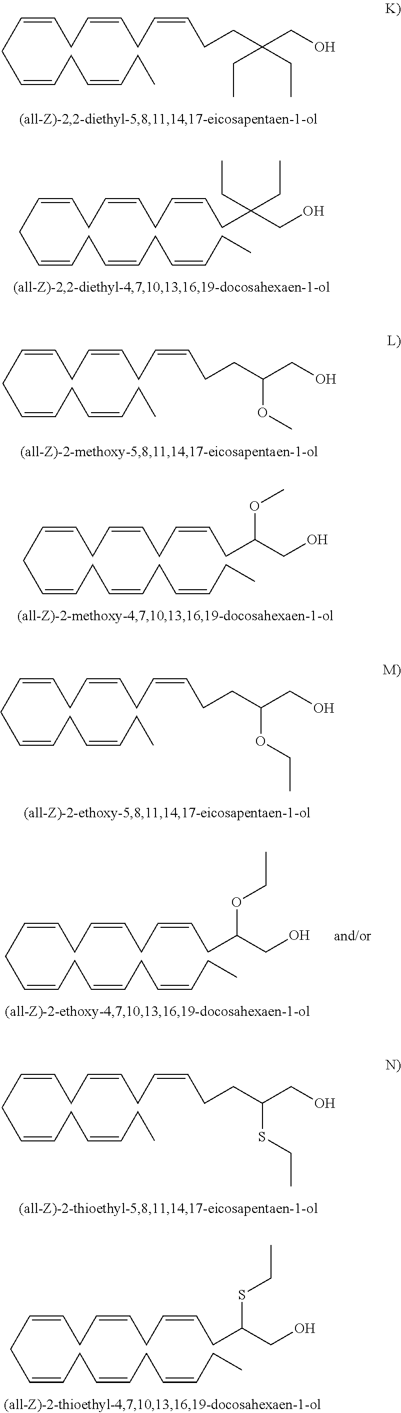

It is to be understood that the present invention encompasses any possible pharmaceutically acceptable complexes, solvates or pro-drugs of the omega-3 lipid compounds of formulas (I) and (II).

An exemplary embodiment of the invention includes a composition comprising at least omega-3 lipid compounds substituted in the 2-position, wherein the omega-3 lipid compounds comprise:

a compound of formula (I):

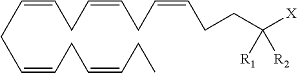

and a compound of formula (II):

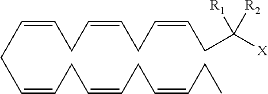

wherein
the weight ratio of compounds of formula (I):compounds of formula (II) is from 1:10 to 10:1, and

wherein
R₁ and R₂ are the same or different and are chosen from methyl, ethyl, propyl, dimethyl, diethyl, thiomethyl, thioethyl, methoxy, ethoxy, OH, methylamino and ethylamino; and
X represents a carboxylic acid or a derivative thereof, a carboxylate, a hydroxymethyl (—CH₂OH) or a pro-drug thereof, or a carboxamide.

Another exemplary embodiment of the invention includes a composition comprising at least omega-3 lipid compounds substituted in the 2-position, wherein the omega-3 lipid compounds comprise:

a compound of formula (I):

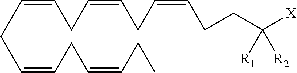

and a compound of formula (II):

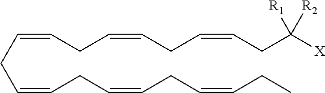

wherein
the weight ratio of compounds of formula (I):compounds of formula (II) is from 1:5 to 5:1, and
R₁ and R₂ are chosen from methyl, ethyl, propyl, ethoxy, methoxy, benzyl, thiomethyl and thioethyl; and
X represents an ethyl carboxylate or a hydroxymethyl (—CH₂OH).

Another exemplary embodiment of the invention includes a composition comprising at least omega-3 lipid compounds substituted in the 2-position, wherein the omega-3 lipid compounds comprise:

a compound of formula (I):

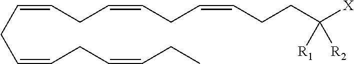

and a compound of formula (II):

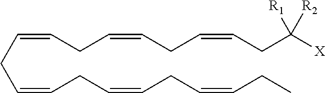

wherein
the compounds of the formula (I) and (II) are present in a concentration of at least about 60% by weight of the total omega-3 lipid compounds, and
R₁ and R₂ are chosen from methyl, ethyl, propyl, ethoxy, methoxy, benzyl, thiomethyl and thioethyl; and
X represents an ethyl carboxylate or a hydroxymethyl (—CH₂OH).

Where X is a carboxylic acid, the present invention also includes salts of the carboxylic acids. Suitable pharmaceutically acceptable salts of carboxyl groups include metal salts, for example alkali metal salts, such as lithium, sodium or potassium, alkaline metal salts, such as calcium or magnesium, and ammonium or substituted ammonium salts. Furthermore, additional salts include substituted ammonium salts, meglumine salt, tris(hydroxymethyl)aminomethane salt, arginine salt, piperazine salt, and Chitosan salt.

A “pharmaceutically active amount” relates to an amount that will lead to the desired pharmacological and/or therapeutic effects, i.e. an amount of the omega-3 lipid compounds according to this invention which is effective to achieve an intended purpose. While individual patient needs may vary, determination of optimal ranges for effective amounts of the omega-3 lipid compounds of this invention is within the skill of the art. Generally, the dosage regimen for treating a condition with the compounds and/or compositions of this invention is selected in accordance with a variety of factors, including the type, age, weight, sex, diet and medical condition of the patient.

By “a medicament” is meant a composition of omega-3 lipid compounds according to formula (I) and (II), in any form suitable to be used for a medical purpose, e.g. in the form of a medicinal product, a pharmaceutical preparation or product, a dietary product, a food stuff or a food supplement.

“Treatment” includes any therapeutic application that can benefit a human or non-human mammal. Both human and veterinary treatments are within the scope of the present invention. Treatment may be with respect to an existing condition or it may be prophylactic.

The omega-3 composition comprising compounds of formula (I) and (II) may be used on its own but will generally be administered in the form of a pharmaceutical composition in which the compounds of formula (I) and (II) (the active ingredients) are in association with a pharmaceutically acceptable carrier, an excipient, a diluent, or a combination thereof.

Acceptable carriers, excipients and diluents for therapeutic use are well known in the pharmaceutical art, and can be selected with regard to the intended route of administration and standard pharmaceutical practice. Examples encompass binders, lubricants, suspending agents, coating agents, solubilising agents, preserving agents, wetting agents, emulsifiers, sweeteners, colorants, flavouring agents, odorants, buffers, suspending agents, stabilising agents, and/or salts.

A pharmaceutical composition according to the invention is preferably formulated for oral administration to a human or an animal. The pharmaceutical composition may also be formulated for administration through any other route where the active ingredients may be efficiently absorbed and utilized, e.g. intravenously, subcutaneously, intramuscularly, intranasally, rectally, vaginally or topically.

In an exemplary embodiment of the invention, the pharmaceutical composition is shaped in form of a capsule, which could also be a microcapsule, generating a powder or a sachet in bulk. The capsule may be flavoured. This embodiment also includes a capsule wherein both the capsule and the encapsulated composition according to the invention is flavoured. By flavouring the capsule it becomes more attractive to the user. For the above-mentioned therapeutic uses the dosage administered will, of course, vary with the compounds employed, the mode of administration, the treatment desired and the disorder indicated.

The pharmaceutical composition may be formulated to provide a daily dosage of e.g. 5 mg to 10 g; 50 mg to 1 g; or 50 mg to 200 g of the composition. By a daily dosage is meant the dosage per a 24 hour period.

The dosage administered will, of course, vary with the compounds employed, the mode of administration, the treatment desired and the disorder indicated. Typically, a physician will determine the actual dosage which will be most suitable for an individual subject. The specific dose level and frequency of dosage for any particular patient may be varied and will depend upon a variety of factors including the activity of the specific compounds employed, the metabolic stability and length of action of those compounds, the age, body weight, general health, sex, diet, mode and time of administration, rate of excretion, drug combination, the severity of the particular condition, and the individual undergoing therapy. The omega-3 lipid compounds and/or the pharmaceutical compositions of the present invention may be administered in accordance with a regimen of from 1 to 10 times per day, such as once or twice per day. For oral and parenteral administration to human patients, the daily dosage level of the agent may be in single or divided doses.

In an exemplary embodiment of the composition according to the present invention the substituents R₁ and R₂ are chosen from methyl, ethyl, propyl, dimethyl, diethyl, thiomethyl, thioethyl, methoxy, ethoxy, OH, methylamino and ethylamino.

In another exemplary embodiment R₁ and R₂ are chosen from methyl, ethyl, propyl, ethoxy, thiomethyl, thioethyl and methoxy. In another exemplary embodiment of the present invention R₁ and R₂ are chosen from ethyl, propyl or ethoxy.

The composition may further comprise at least one of (all-Z)-6,9,12,15,18-heneicosapentaenoic acid (H PA), and (all-Z)-7,10,13,16,19-docosapentaenoic acid (DPAn-3), (all-Z)-8,11,14,17-eicosatetraenoic acid (ETAn-3), or combinations thereof, substituted in their alpha position. Further, a composition may comprise (all-Z)-4,7,10,13,16-Docosapentaenoic acid (DPAn-6) and/or (all-Z)-5,8,11,14-eicosatetraenoic acid (ARA), or derivatives thereof. The composition may also comprise at least these fatty acids, or combinations thereof, in the form of derivatives. The derivatives may be suitably substituted in the same way as the EPA and DHA derivatives constituting the composition according to the invention.

The composition comprising at least one compound of formula (I) and a compound of formula (II) has pharmaceutical activity, in particular it triggers nuclear receptor activity. Thus, the present invention also relates to said compositions, pharmaceutically acceptable salts, solvates, complexes or pro-drugs thereof, as hereinbefore defined, for use as a medicament and/or for use in therapy. Preferably, the novel compositions, or pharmaceutically acceptable salts, solvates, complexes or pro-drugs thereof, of the invention may be used:
for the prevention and/or treatment of diabetes mellitus in humans or animals;
for controlling body weight reduction and/or for preventing body weight gain in humans or animals;
for the prevention and/or treatment of obesity or an overweight condition in humans or in an animal;
for the treatment and/or prevention of amyloidos-related diseases;
for the treatment or prophylaxis of multiple risk factors for cardiovascular diseases;
for the prevention of stroke, cerebral or transient ischaemic attacks related to atherosclerosis of several arteries.
for the treatment of TBC or HIV.

There are two major forms of diabetes mellitus. One is type 1 diabetes, which is known as insulin-dependent diabetes mellitus (IDDM), and the other one is type 2 diabetes, which is also known as non-insulin-dependent diabetes mellitus (NIDDM). Type 2 diabetes is related to obesity/overweight and lack of exercise, often of gradual onset, usually in adults, and caused by reduced insulin sensitivity, so called peripheral insulin resistance. This leads to a compensatory increase in insulin production. This stage before developing full fledged type 2 diabetes is called the metabolic syndrome and characterized by hyperinsulinemia, insulin resistance, obesity, glucose intolerance, hypertension, abnormal blood lipids, hypercoagulopathia, dyslipidemia and inflammation, often leading to atherosclerosis of the arteries. Later when insulin production ceases, type 2 diabetes mellitus develops.

In an exemplary embodiment, the compositions comprising compounds of formula (I) and formula (II) may be used for the treatment of type 2 diabetes. The said compositions may also be used for the treatment of other types of diabetes chosen from metabolic syndrome, secondary diabetes, such as pancreatic, extrapancreatic/endocrine or drug-induced diabetes, or exceptional forms of diabetes, such as lipoatrophic, myatonic or a disease caused by disturbance of the insulin receptors. The invention also includes treatment of type 2 diabetes. Suitably, compositions according to the invention, as hereinbefore defined, may activate nuclear receptors, preferably PPAR (peroxisome proliferator-activated receptor) α and/or γ.

The compositions comprising at least one compound of formula (I) and a compound of formula (II) may also be used for the treatment and/or prevention of obesity. Obesity is usually linked to an increased insulin resistance and obese people run a high risk of developing type 2 diabetes which is a major risk factor for development of cardiovascular diseases. Obesity is a chronic disease that afflicts an increasing proportion of the population in Western societies and is associated, not only with a social stigma, but also with decreasing life span and numerous problems, for instance diabetes mellitus, insulin resistance and hypertension.

The compositions comprising at least one compound of formula (I) and a compound of formula (II) may also be used for the prevention and/or treatment of amyloidos-related diseases. Amyloidos-related conditions or diseases associated with deposition of amyloid, preferably as a consequence of fibril or plaque formation, includes Alzheimer's disease or dementia, Parkinson's disease, amyotropic lateral sclerosis, the spongiform encephalopathies, such as Creutzfeld-jacob disease, cystic fibrosis, primary or secondary renal amyloidoses, IgA nephropathy, and amyloid deposition in arteries, myocardium and neutral tissue. These diseases can be sporadic, inherited or even related to infections such as TBC or HIV, and are often manifested only late in life even if inherited forms may appear much earlier. Each disease is associated with a particular protein or aggregates of these proteins, which are thought to be the direct origin of the pathological conditions associated with the disease. The treatment of a amyloidos-related disease can be made either acutely or chronically.

Compositions according to the invention may also be used for the treatment due to reduction of amyloid aggregates, prevention of misfolding of proteins that may lead to formation of so called fibrils or plaque, treatment due to decreasing of the formation of so called fibrils or plaque, treatment due to decreasing of the production of precursor protein such as Aβ-protein (amyloid beta protein), and prevention and/or treatment due to inhibiting or slowing down of the formation of protein fibrils, aggregates, or plaque. Prevention of fibril accumulation, or formation, by administering compounds of formula (I), as hereinbefore defined, is also included herein. In one embodiment, the novel compositions, pharmaceutically acceptable salts, solvates, complexes or pro-drugs thereof, as hereinbefore defined, are used for the treatment of TBC (tuberculosis) or HIV (human immunodeficiency virus).

Further, the compositions according to the invention may be administered to patients with symptoms of atherosclerosis of arteries supplying the brain, for instance a stroke or transient ischaemic attack, in order to reduce the risk of a further, possible fatal, attack.

The compositions according to the invention may also be used for the treatment of elevated blood lipids in humans.

Additionally, the compositions according to the invention, as hereinbefore defined, are valuable for the treatment and prophylaxis of multiple risk factors known for cardiovascular diseases, such as hypertension, hypertriglyceridemia and high coagulation factor VII phospholipid complex activity. Preferably, the present composition is used for the treatment of elevated blood lipids in humans.

The composition comprising compounds of formula (I) and (II) and pharmaceutically acceptable salts, solvates, pro-drugs or complexes thereof may be used on their own but will generally be administered in the form of a pharmaceutical composition in which the compounds of formula (I) and formula (II) (the active ingredients) are in association with a pharmaceutically acceptable adjuvant, diluent or carrier.

Acceptable carriers or diluents for therapeutic use are well known in the pharmaceutical art. The choice of pharmaceutical carrier, excipient or diluent can be selected with regard to the intended route of administration and standard pharmaceutical practice. The pharmaceutical compositions may comprise as- or in addition to- the carrier, excipient or diluent any suitable binder(s), lubricant(s), suspending agent(s), coating agent(s), solubilising agent(s).

Pharmaceutical compositions within the scope of the present invention may include one or more of the following: preserving agents, solubilising agents, stabilising agents, s wetting agents, emulsifiers, sweeteners, colorants, flavouring agents, odorants, salts compounds of the present invention may themselves be provided in the form of a pharmaceutically acceptable salt), buffers, coating agents, antioxidants, suspending agents, adjuvants, excipients and diluents.

A pharmaceutical composition according to the invention is preferably formulated for oral administration to a human or an animal. The pharmaceutical composition may also be formulated for administration through any other route where the active ingredients may be efficiently absorbed and utilized, e.g. intravenously, subcutaneously, intramuscularly, intranasally, rectally, vaginally or topically.

In an exemplary embodiment of the invention, the pharmaceutical composition is shaped in the form of a capsule, which could also be microcapsules generating a powder or a sachet. The capsule may be flavoured. The invention includes a capsule wherein both the capsule and the encapsulated fatty acid composition according to the invention is flavoured. By flavouring the capsule it becomes more attractive to the user. For the above-mentioned therapeutic uses the dosage administered will, of course, vary with the compound employed, the mode of administration, the treatment desired and the disorder indicated.

The pharmaceutical composition may be formulated to provide a daily dosage of 10 mg to 10 g. Preferably, the pharmaceutical composition is formulated to provide a daily dosage between 50 mg and 5 g of said composition. Most preferably, the pharmaceutical composition is formulated to provide a daily dosage between 100 mg and 1 g of said composition. By a daily dosage is meant the dosage per 24 hours.

In some embodiments of the invention, the composition is a pharmaceutical composition, a nutritional composition, or a dietary composition.

The composition may further comprise an effective amount of a pharmaceutically acceptable antioxidant. Preferably, the antioxidant is tocopherol or a mixture of tocopherols, or an astaxanthin. In an exemplary embodiment the composition further comprises tocopherol, or a mixture of tocopherols, in an amount of up to 4 mg per g of the total weight of the composition. Preferably, the composition comprises an amount of 0.2 to 0.4 mg per g of tocopherols, based on the total weight of the composition.

Another aspect of the invention provides a composition, or any pharmaceutically acceptable salt, solvate, pro-drug or complex thereof, comprising compounds of formula (I) and (II), as hereinbefore defined, for use as a medicament and/or in therapy. When the composition is used as a medicament, it will be administered in a therapeutically or a pharmaceutically active amount.

In an exemplary embodiment, the composition is administered orally to a human or an animal.

The present invention also provides the use of a composition comprising at least one compound of formula (I) and one compound of formula (II), or pharmaceutically acceptable salts, solvates, pro-drugs or complexes thereof, as hereinbefore defined, for the manufacture of a medicament for controlling body weight reduction and/or for preventing body weight gain; for the manufacture of a medicament for the treatment and/or the prevention of obesity or an overweight condition; for the manufacture of a medicament for the prevention and/or treatment of diabetes in a human or animal; for the manufacture of a medicament for the treatment and/or prevention of amyloidos-related diseases; for the manufacture of a medicament for the treatment and prophylaxis of multiple risk factors known for cardiovascular diseases, such as hypertension, hypertriglyceridemia and high coagulation factor VII phospholipid complex activity; for the manufacture of a medicament for the treatment of TBC or HIV; for the manufacture of a medicament for prevention of stroke, cerebral or transient ischaemic attacks related to atherosclerosis of several arteries; for the manufacturing of a medicament for lowering triglycerides in the blood of mammals and/or increasing the HDL cholesterol levels in the serum of a human patients; or for the manufacturing of a medicament for the treatment and/or prevention of the multi metabolic syndrome termed “metabolic syndrome”. All these embodiments also include the use of a composition, as hereinbefore defined, comprising compounds of formula (I) and compounds of formula (II) for the manufacture of medicaments as outlined above.

The present invention also relates to a method for controlling body weight reduction and for preventing body weight gain, wherein a composition comprising at least a compound of formula (I) and a compound of formula (II), as hereinbefore defined, is administered to a human or an animal.

Further, the invention relates to a method for the treatment and/or the prevention of obesity or an overweight condition, wherein a composition comprising at least a compound of formula (I) and a compound of formula (II), as hereinbefore defined, is administered to a human or an animal.

In a preferred embodiment of the invention, the present invention relates to a method for the prevention and/or treatment of diabetes mellitus, wherein a composition comprising at least a compound of formula (I) and a compound of formula (II), as hereinbefore defined, is administered to a human or an animal. Preferably, diabetes mellitus is a type 2 diabetes.

Finally, the invention also relates to methods for the manufacture of a composition according to the invention. Preferably, said composition is prepared from a vegetable, a microbial and/or an animal source. More preferably, the composition according to the invention is prepared from a fish oil, or a krill oil.

Methods

A composition according to the invention may be prepared from a composition comprising EPA and DHA in addition to other PUFAs that are obtained from a vegetable, microbial, algae or a marine source or combinations thereof. The compositions according to the invention may also be prepared by mixing α-substituted PUFA derivatives in the desired composition. Preferably the PUFA composition is obtained from a marine source such as fish oil, krill oil, seal oil.

Methods for Preparing the Compounds According to the Invention

The omega-3 lipid compound of formula (I) where R₁ (or R₂) is a hydrogen may be prepared through the following processes (Scheme 1). Omega-3 lipid compounds represented by the general formula (I) where R₁ is a hydrogen and R₂ denotes a C₁-C₆ alkyl group, a benzyl, a halogen, a benzyl, an alkenyl, an alkynyl are prepared by reacting a long chain polyunsaturated ester with a strong non-nucleophilic base like lithium diisopropylamine, potassium/sodium hexamethyldisilazide or KH/NaH in DMF in a solvent such as tetrahydrofuran, diethylether at temperatures of −60 to −78° C., to provide the ester enolate (process 1).

Method I

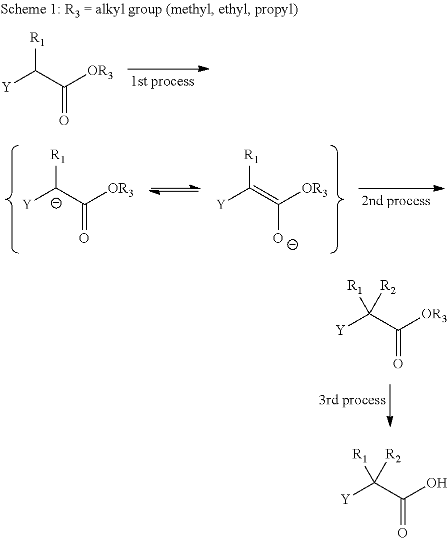

This ester enolate is reacted with an electrophilic reagent like an alkylhalide exemplified by ethyliodine, benzylcloride, an acyl halide exemplified by; acetyl chloride, benzoyl bromide, a carboxylic anhydride exemplified by acetic anhydride or a electrophilic halogenation reagent exemplified by N-fluorobenzene sulfonimide (NFSI), N-bromosuccinimide or iodine etc. to provide the substituted derivative (process 2). The 2-halo substituted derivatives can be reacted with a nucleophilic reagent such as tiols to provide 2-alkylthio-derivatives.

The ester is further hydrolysed in a solvent like ethanol or methanol to the carboxylic acid derivative by addition of a base like lithium/sodium/potassium hydroxide in water at temperatures between 15° C. and reflux.

Claisen condensation of the long chain polyunsaturated ester occurs during the treatment of ester with a strong base. (This condensation product might possess interesting biologically activity. Thus, in one embodiment of the invention the condensation (intermediate) product mentioned above, as well as the use of this product for treatment and/or prevention of diseases according to the present invention, are disclosed.)

Moreover, in a further embodiment, compounds represented by the general formula (I) are synthesised through following processes (Scheme 2).

Method II:

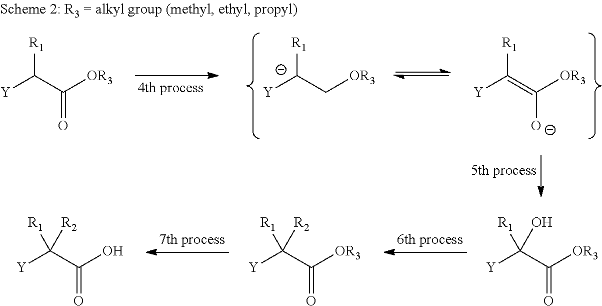

Compounds represented by the general formula (I) where R₁ is a hydrogen and R₂ denotes a hydroxy, an alkoxy group, an acyloxy are prepared by reacting a long chain polyunsaturated ester with a strong non-nucleophilic base like lithium diisopropylamine or potassium/sodium hexamethyldisilazide in a solvent such as tetrahydrofuran, diethylether at temperatures of −60 to −78° C., to provide the ester enolate (process 4). This ester enolate is reacted with an oxygen source like dimethyldioxirane, 2-(phenylsulfonyl)-3-phenyloxaziridine, molecular oxygen with different additives like trimethylphosphite or different catalysts like a Ni(II) complex to provide alpha-hydroxy ester (process 5). Reaction of the secondary alcohol with a base like sodiumhydride in a solvent like THF or DMF generates an alkoxide that is reacted with different electrophilic reagents as alkyliodide for example; methyl iodide, ethyl iodide, benzylbromide or an acyl halide, for example; acetyl chloride, benzoyl bromide (process 6). The ester is hydrolysed in a solvent like ethanol or methanol to the carboxylic acid derivative by addition of a base like lithium/sodium/potassium hydroxide in water at temperatures between 15° C. to reflux (process 7).

The alpha-hydroxy ester is a useful intermediate for the introduction of other functional groups in the α-position according to the invention. The hydroxyl function can be activated by conversion to a halide or tosylate prior to reaction with different nucleophiles like ammonia, amines, thiols, etc. The Mitsunobu reaction is also useful for the conversion of a hydroxyl group into other functional groups. (Mitsunobu, O, Synthesis, 1981, 1).

### Examples Synthesis

The examples below illustrate the preparation of the composition according to the invention. They are, however, not to be construed as a limitations to the scope thereof.

In these examples a lipid mixture containing 90% omega-3 PUFAs as ethylesters was used as starting material. The mixture contained approximately 85% w/w of ethyl (all-Z)-5,8,11,14,17-eicosapentaenoate and ethyl (all-Z)-4,7,10,13,16,19-docosahexaenoate in a ratio of 1.2 w/w. For simplicity this mixture is called 85/EPA/DHA-EE. Other PUFA ethylester mixtures can be used as starting materials.

Preparation of α-ethyl 85/EPA/DHA-EE:

Butyllithium (3.9 ml, 6.3 mmol, 1.6 M in hexane) was added dropwise to a stirred solution of diisopropylamine (0.93 ml, 6.6 mmol) in dry THF (10 ml) under N₂ at 0° C. The resulting solution was stirred at 0° C. for 20 min., cooled to −78° C. and stirred an additional 10 min. before dropwise addition of 85/EPA/DHA-EE (2.0 g, 5.7 mmol) in dry THF (10 mL) during 10 min. The green solution was stirred at −78° C. for 10 min. before ethyl iodide (0.69 ml, 8.6 mmol) was added. The resulting solution was allowed to reach ambient temperature over one hour, portioned between water (40 mL) and heptane (40 mL). The aqueous layer was extracted with heptane (40 mL) and the combined organic layer was washed with 1M HCl (40 mL) and dried (Na₂SO₄). Concentration under reduced pressure and purification by flash chromatography (Heptane: EtOAc 98:2) afforded 1.53 g (68%) of the title compound as mixture of ethyl (all-Z)-2-ethyl-5,8,11,14,17-eicosapentaenoate [α-ethyl EPA EE] and ethyl (all-Z)-2-ethyl-4,7,10,13,16,19-docosahexaenoate [α-ethyl DHA EE] as a colorless oil; ¹H-NMR (200 MHz, CDCl₃): δ 0.84-0.99 (m, 7H), 1.12-1.28 (m, 5H), 1.40-1.80 (m, 4H), 2.02-2.09 (m, 3H), 2.27 (m, 1H), 2.70-2.90 (m, 9H), 4.13 (q, 2H), 5.28-5.44 (m, 11H); MS (electrospray): 381.2 [α-ethyl EPA EE+Na], 407.2 [α-ethyl DHA EE+Na].

Reduction of α-ethyl 85/EPA/DHA-EE:

A suspension of LAH (0.054 g, 1.42 mmol) in dry THF (5 mL) under inert atmosphere was given 0° C. and α-ethyl 85/EPA/DHA-EE (0.50 g, 1.35 mmol) in dry THF (5 mL) was added dropwise. The mixture was stirred at 0° C. for 30 minutes, added 10% NH₄Cl (10 mL) and filtrated through a short pad of celite. The pad was washed with water (20 mL) and heptane (20 mL) and the layers were separated. The aqueous phase was extracted with heptane (20 mL) and the combined organic layer was washed with brine (20 mL) and dried (MgSO₄). This afforded 0.35 g (79%) of the title compound as a 1.2:1 mixture of α-ethyl EPA-OH and α-ethyl DHA-OH, as a colorless oil. ¹H-NMR (200 MHz, CDCl₃): δ 0.86-0.99 (m, 8H), 1.32-1.41 (m, 6H), 1.98-2.12 (m, 4H), 2.80-2.90 (m, 10H), 3.51-3.55 (m, 2H), 5.26-5.43 (m, 12H); ¹³C-NMR (50 MHz, CDCl₃): δ 11.03, 11.32, 14.05, 14.21, 20.50, 22.64, 23.23, 23.36, 24.56, 25.48, 25.58, 28.45, 30.36, 31.83, 41.50, 42.53, 64.96, 65.13, 126.96, 127.76, 127.82, 127.97, 128.05, 128.07, 128.09, 128.15, 128.17, 128.22, 128.34, 128.51, 129.03, 130.21, 131.97; MS (electrospray): 339.2 [α-ethyl EPA-OH+Na], 365.3 [α-ethyl DHA-OH+Na].

Formulations and Compositions

Processes for the fractionation of polyunsaturated fatty acids or polyunsaturated fatty acid alkyl esters from marine oils may be carried out separately or combined in order to produce mixed-fatty acid compositions with concentrations of EPA and DHA varying over a wide range, and the samples available commercially reflect this. The concentrations of EPA and DHA depend on the concentration in the starting material and the fractionation process used, as well as the process yield.

Fractionation of polyunsaturated fatty acids from marine oils by short path distillation or supercritical fluid fractionation commonly produces long-chain polyunsaturated omega-3 oils with a concentration of EPA+DHA of 50-60% by weight, typically containing 30-40% EPA and 20-30% DHA. Commercial examples of such mixed-fatty acid compositions are EPAX5500TG and EPAX6000FA (EPAX A.S.), K50EE (Pronova Biocare A.S.), Incromega E3322 and Incromega TG3322 (Croda), and MEG-3 Concentrate 30/20 EE and MEG-3 Concentrate 40/20 TG (Ocean Nutrition Canada). These compositions comprising at least EPA and DHA may be;
substituted in their alpha position, and
in the form of an alcohol or and ester according to the invention.

Particular fractionation may be carried out in order to produce high purity long-chain polyunsaturated omega-3 oils, typically EPA+DHA>75%. Commercial examples of such mixed-fatty acid compositions are K70EE, K80EE, K85EE, K85TG, and AGP103 (Pronova Biocare A.S.). Also these compositions comprising at least EPA and DHA, and may be;
substituted in their alpha position, and
in the form of an alcohol or and ester according to the invention.

Moreover, fractionation of polyunsaturated fatty acids or ethyl esters may be carried out in such a way as to manufacture long-chain polyunsaturated omega-3 oils which are selectively enriched in EPA. Commercial examples of such mixed-fatty acid compositions are EPAX4510TG and EPAX7010EE (EPAX A.S.), Incromega EPA500TG and Incromega E7010 SR (Croda), and MEG-3 60/03TG and MEG-3 50/20EE (Ocean Nutrition Canada), Such products are also included herein;
substituted in their alpha position, and
being in the form of an alcohol or and ester according to the invention.

Additionally, fractionation of fatty acids or fatty acid ethyl esters may be carried out in such a way as to manufacture long-chain omega-3 oils which are selectively enriched in DHA. Commercial examples of such mixed-fatty acid compositions are EPAX2050TG (EPAX A.S.), Incromega DHA500TG and Incromega 700E SR (Croda), and MEG-3 20/50TG and MEG-3 05/55EE (Ocean Nutrition Canada).

Thus, all commercial examples mentioned could be substituted in the 2-position according to the general methods outlined above and well-known in the art. These compounds may be present as their alcohols or ethyl esters.

The invention shall not be limited to the shown embodiments or examples.

1. A lipid composition comprising omega-3 lipid compounds substituted at carbon 2, counted from the functional group of the omega-3 lipid compound,
which omega-3 lipid compounds comprise
a compound of the general formula (I): 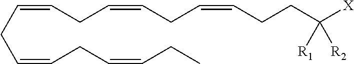
and a compound of the general formula (II): 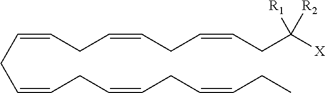
wherein
R₁ and R₂ are the same or different and are chosen from hydrogen, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; and
X is chosen from a carboxylic acid or a derivative thereof, a carboxylate, a carboxylic anhydride, a hydroxymethyl (—CH₂OH) or a pro-drug thereof, and a carboxamide,
or any pharmaceutically acceptable complex, salt, solvate or pro-drug, with the proviso that:
R₁ and R₂ are not simultaneously a hydrogen atom.

2. A lipid composition according to claim 1, wherein the omega-3 lipid compounds substituted at carbon 2, are present in a concentration of at least 30% by weight as compared to the total lipid content of the composition.

3. A lipid composition according to claim 1, wherein the omega-3 lipid compounds substituted at carbon 2, are present in a concentration of at least 50% by weight as compared to the total lipid content of the composition.

4. A lipid composition according to claim 1, wherein the omega-3 lipid compounds substituted at carbon 2 are present in a concentration of at least 70% by weight as compared to the total lipid content of the composition.

5. A lipid composition according to claim 1, wherein the omega-3 lipid compounds substituted at carbon 2, are present in a concentration of at least 80% by weight as compared to the total lipid content of the composition.

6. A lipid composition according to claim 1, wherein the compounds of the general formula (I) and formula (II) comprise at least about 20% by weight of omega-3 lipid compounds substituted at carbon 2.

7. A lipid composition according to claim 1, wherein the compounds of the general formula (I) and formula (II) comprise at least about 40% by weight of omega-3 lipid compounds substituted at carbon 2.

8. A lipid composition according to claim 1, wherein the compounds of the general formula (I) and formula (II) comprise at least about 70% by weight of omega-3 lipid compounds substituted at carbon 2.

9. A lipid composition according to claim 1, wherein the compounds of the general formula (I) and formula (II) comprise at least about 80% by weight of omega-3 lipid compounds substituted at carbon 2.

10. A lipid composition according to claim 1, wherein the compounds of formula (I) are present in a concentration of about 5% to 95% by weight of the total lipid content in the composition.

11. A lipid composition according to claim 10, wherein the compounds of formula (I) are present in a concentration of about 40% to 55% by weight of the total lipid content in the composition.

12. A lipid composition according to claim 1, wherein the compounds of formula (II) are present in a concentration of about 5% to 95% by weight of the total lipid content in the composition.

13. A lipid composition according to claim 12, wherein the compounds of formula (II) are present in a concentration of about 30% to 60% by weight of the total lipid content in the composition.

14. A lipid composition according to claim 1, wherein the omega-3 lipid compounds substituted at carbon 2, comprise compounds of formula (I) and formula (II) in a weight ratio of [compounds of formula (I)]:[compounds of formula (II)] from 1:99 to 1:99.

15. A lipid composition according to claim 14, wherein the omega-3 lipid compounds substituted at carbon 2, comprise compounds of formula (I) and formula (II) in a weight ratio of [compounds of formula (I)]:[compounds of formula (II)] from 10:1 to 1:10.

16. A lipid composition according to claim 15, wherein the omega-3 lipid compounds substituted at carbon 2, comprise compounds of formula (I) and formula (II) in a weight ratio of [compounds of formula (I)]:[compounds of formula (II)] from 5:1 to 1:5.

17. A lipid composition according to claim 16, wherein the omega-3 lipid compounds substituted at carbon 2, comprise compounds of formula (I) and formula (II) in a weight ratio of [compounds of formula (I)]:[compounds of formula (II)] from 3:1 to 1:3.

18. A lipid composition according to claim 17, wherein the omega-3 lipid compounds substituted at carbon 2, comprise compounds of formula (I) and formula (II) in a weight ratio of [compounds of formula (I)]:[compounds of formula (II)] from 1.2 to 2.1.

19. A lipid composition according to claim 1, wherein said alkyl group is chosen from methyl, ethyl, n-propyl, isopropyl, n-butyl, iso-butyl, sec-butyl, and n-hexyl.

20. A lipid composition according to claim 1, wherein said halogen atom is fluorine.

21. A lipid composition according to claim 1, wherein said alkoxy group is chosen from methoxy, ethoxy, propoxy, isopropoxy, sec-butoxy, phenoxy, benzyloxy, OCH₂CF₃, and OCH₂CH₂OCH₃.

22. A lipid composition according to claim 1, wherein said alkynyl groups is chosen from allyl, 2-butenyl, and 3-hexenyl.

23. A lipid composition according to claim 1, wherein said alkynyl group is chosen from propargyl, 2-butynyl, and 3-hexynyl.

24. A lipid composition according to claim 1, wherein said aryl group is a benzyl or substituted benzyl group.

25. A lipid composition according to claim 1, wherein said alkylthio group is chosen the methylthio, ethylthio, isopropylthio, and phenylthio.

26. A lipid composition according to claim 1, wherein said alkoxycarbonyl group is chosen from methoxycarbonyl, ethoxycarbonyl, propoxycarbonyl, and butoxycarbonyl.

27. A lipid composition according to claim 1, wherein said alkylsulfinyl group chosen from methanesulfinyl, ethanesulfinyl, and isopropanesulfinyl.

28. A lipid composition according to claim 1, wherein said alkylsulfonyl group is chosen from methanesulfonyl, ethanesulfonyl, and isopropane sulfonyl.

29. A lipid composition according to claim 1, wherein said alkylamino is chosen from methylamino, dimethylamino, ethylamino, and diethylamino.

30. A lipid composition according to claim 1, wherein said carboxylate group is chosen from ethyl carboxylate, methyl carboxylate, n-propyl carboxylate, isopropyl carboxylate, n-butyl carboxylate, sec-butyl carboxylate, and hexyl carboxylate.

31. A lipid composition according to claim 1, wherein said carboxamide group is chosen from a primary carboxamide, N-methyl carboxamide, N,N-dimethyl carboxamide, N-ethyl carboxamide, and N,N-diethyl carboxamide.

32. A lipid composition according to claim 1, wherein X is a carboxylic derivative chosen from a mono-, di-, or triglyceride, and a phospholipid.

33. A lipid composition according to claim 1, wherein the salts of the compounds of formulas (I) and (II) are represented by 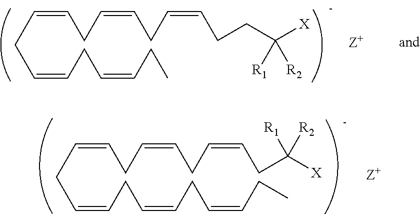
wherein X is COO⁻,
Z⁺ is chosen from Li⁺, Na⁺, K⁺, NH₄⁺, 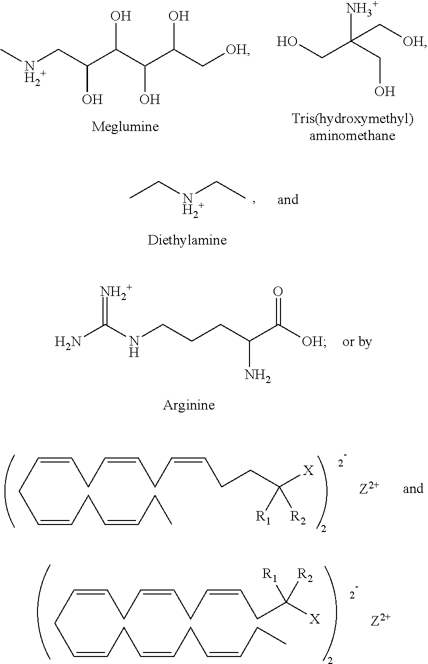
wherein X═COO⁻,
Z²⁺ is chosen from Mg²⁺, Ca²⁺, 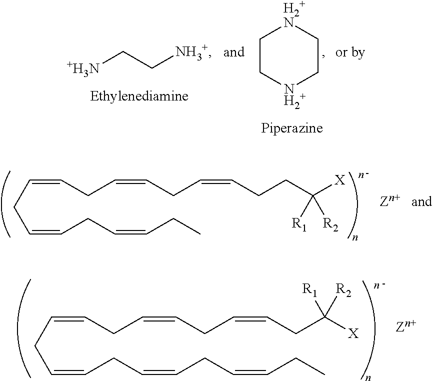
wherein X is COO⁻,
Zⁿ⁺ is

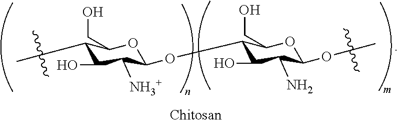

34. A lipid composition according to claim 1, wherein R₁ and R₂ are chosen from hydrogen, a hydroxy group, an alkyl group, a halogen atom, an alkoxy group, an alkylthio group, an alkylsulfinyl group, an alkylsulfonyl group, an amino group, and an alkylamino group.

35. A lipid composition according to claim 33, wherein R₁ and R₂ are chosen from hydrogen, a hydroxy group, a C₁-C₇ alkyl group, a halogen atom, a C₁-C₇ alkoxy group, a C₁-C₇ alkyltio group, a C₁-C₇ alkylsulfinyl group, a C₁-C₇ alkylsulfonyl group, an amino group, and a C₁-C₇ alkylamino group.

36. A lipid composition according claim 35, wherein
said C₁-C₇ alkyl group is methyl, ethyl, or propyl;
said halogen atom is fluorine;
said C₁-C₇ alkoxy group is methoxy or ethoxy;
said C₁-C₇ alkylthio group is methylthio, ethylthio, or phenylthio;
said C₁-C₇ alkylsulfinyl group is ethanesulfinyl;
said C₁-C₇ alkylsulfonyl group is ethanesulfonyl;
said C₁-C₇ alkylamino group is ethylamino or diethylamino; and
X represents an ethyl carboxylate or a carboxamide group.

37. A lipid composition according to claim 1, wherein R₁ and R₂ are chosen from hydrogen, a C₂-C₇ alkyl group, a halogen atom, a C₁-C₇ alkoxy group, a C₁-C₇ alkyltio group, a C₁-C₇ alkylsulfinyl group, a C₁-C₇ alkylsulfonyl group, an amino group, and a C₁-C₇ alkylamino group; and
X represents a hydroxymethyl (—CH₂OH).

38. A lipid composition according claim 37, wherein
said C₂-C₇ alkyl group is methyl, ethyl, or propyl;
said halogen atom is fluorine;
said C₁-C₇ alkoxy group is methoxy or ethoxy;
said C₁-C₇ alkylthio group is methylthio, ethylthio, or phenylthio;
said C₁-C₇ alkylsulfinyl group is ethanesulfinyl;
said C₁-C₇ alkylsulfonyl group is ethanesulfonyl;
said C₁-C₇ alkylamino group is ethylamino or diethylamino;
said acyl is a benzyl;
and X represents hydroxymethyl (—CH₂OH), or 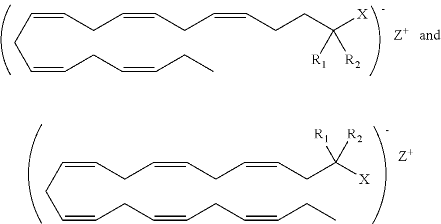
wherein X is COO⁻,
Z⁺ is chosen from Li⁺, Na⁺, K⁺, NH₄⁺, 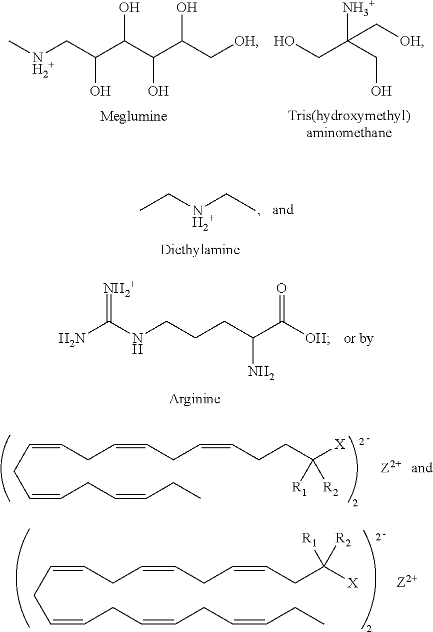
wherein X═COO⁻,
Z²⁺ is chosen from Mg²⁺, Ca²⁺, 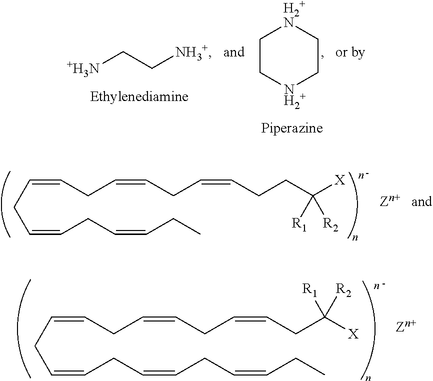
wherein X is COO⁻,
Zⁿ⁺ is

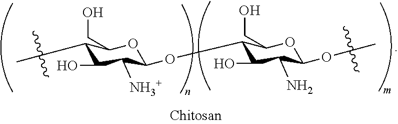

39. A lipid composition according to claim 1, wherein one of R₁ and R₂ is methyl and the other one is a hydrogen.

40. A lipid composition according claim 1, wherein one of R₁ and R₂ is ethyl and the other one is a hydrogen.

41. A lipid composition according to claim 1, wherein one of R₁ and R₂ is propyl and the other one is a hydrogen.

42. A lipid composition according to claim 1, wherein one of R₁ and R₂ is methoxy and the other one is a hydrogen.

43. A lipid composition according to claim 1, wherein one of R₁ and R₂ is ethoxy and the other one is a hydrogen.

44. A lipid composition according to claim 1, wherein one of R₁ and R₂ is propoxy and the other one is a hydrogen.

45. A lipid composition according to claim 1, wherein one of R₁ and R₂ is thiomethyl and the other one is a hydrogen.

46. A lipid composition according to claim 1, wherein one of R₁ and R₂ is thioethyl and the other one is a hydrogen.

47. A lipid composition according to claim 1, wherein one of R₁ and R₂ is thiopropyl and the other one is a hydrogen.

48. A lipid composition according to claim 1, wherein one of R₁ and R₂ is ethylamino and the other one is a hydrogen.

49. A lipid composition according to claim 1, wherein one of R₁ and R₂ is diethylamino and the other one is a hydrogen.

50. A lipid composition according to claim 1, wherein one of R₁ and R₂ is amino and the other one is a hydrogen.

51. A lipid composition according to claim 1, wherein X is ethyl carboxylate or a hydroxylmethyl.

52. A lipid composition comprising at least omega-3 lipid compounds substituted at carbon 2, counted from the functional group of the omega-3 lipid compound,
which omega-3 lipid compounds comprising at least:
a compound of the general formula (I): 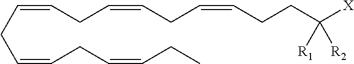
and a compound of the general formula (II): 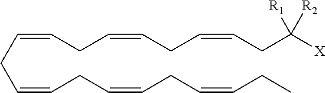
wherein
the ratio of the constituents of formula (I) and (II) in the composition is 1:10 to 10:1, and
wherein
R₁ and R₂ are the same or different and are chosen from methyl, ethyl, propyl, dimethyl, diethyl, thiomethyl, thioethyl, methoxy, ethoxy, OH, methylamino, and ethylamino; and
X is chosen from a carboxylic acid or a derivative thereof, a carboxylate, a carboxylic acid, a hydroxymethyl (—CH₂OH) or a pro-drug thereof, and a carboxamide.

53. A lipid composition according to claim 52, wherein said pro-drugs are present in the form of a pivaloate ester, or a hemisuccinate ester or a salt thereof.

54. A lipid composition comprising at least omega-3 lipid compounds substituted at carbon 2, counted from the functional group of the omega-3 lipid compound,
which omega-3 lipid compounds comprising at least:
a compound of the general formula (I): 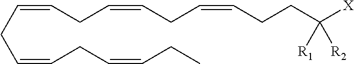
and a compound of the general formula (II): 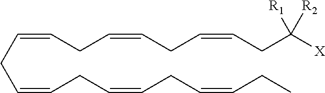
wherein
R₁ and R₂ are chosen from methyl, ethyl, propyl, ethoxy, methoxy, benzyl, thiomethyl, and thioethyl; and
X represents a hydroxymethyl (—CH₂OH).

55. A lipid composition comprising at least omega-3 lipid compounds substituted at carbon 2, counted from the functional group of the omega-3 lipid compound,
which omega-3 lipid compounds comprising at least one of the following combinations of compounds of formula (I) and formula (II);

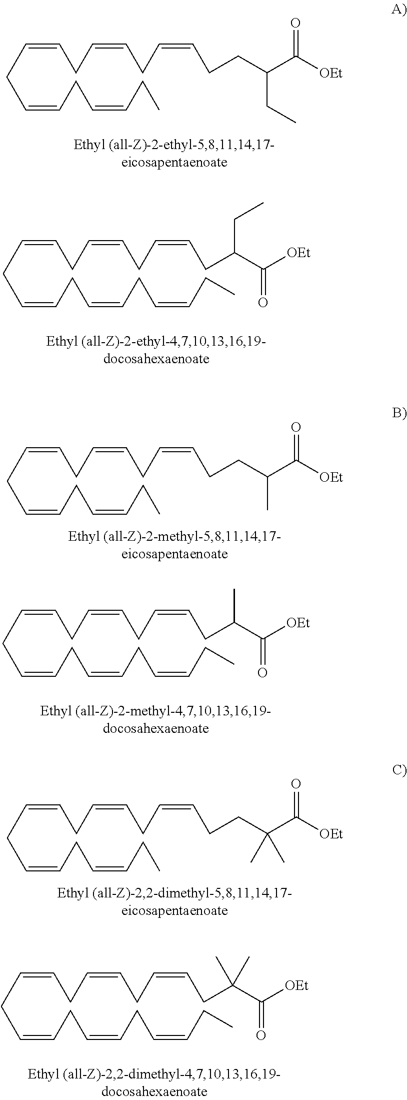

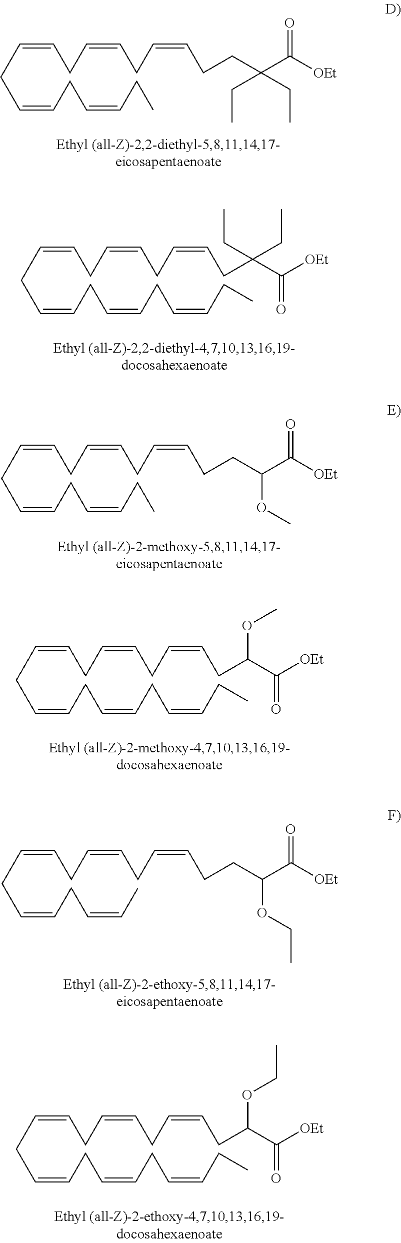

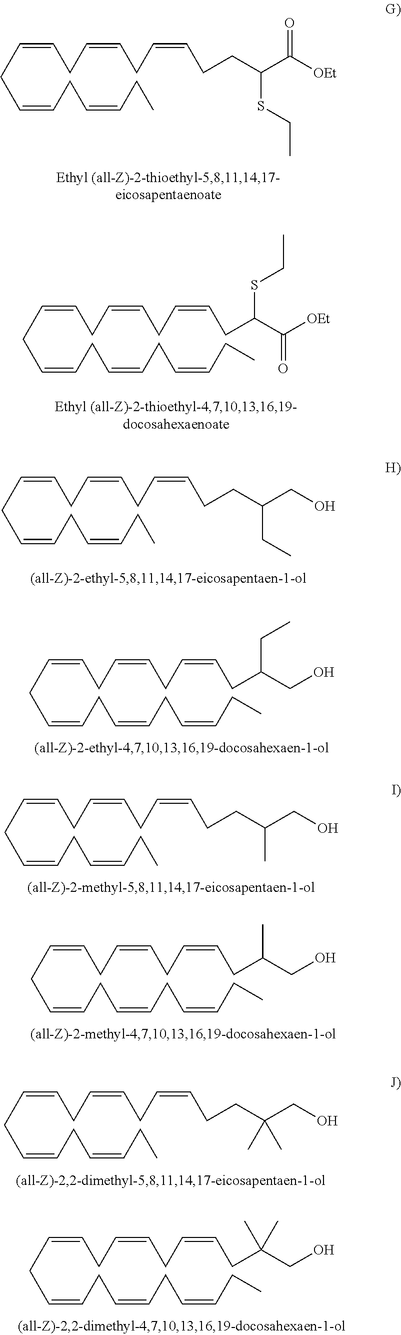

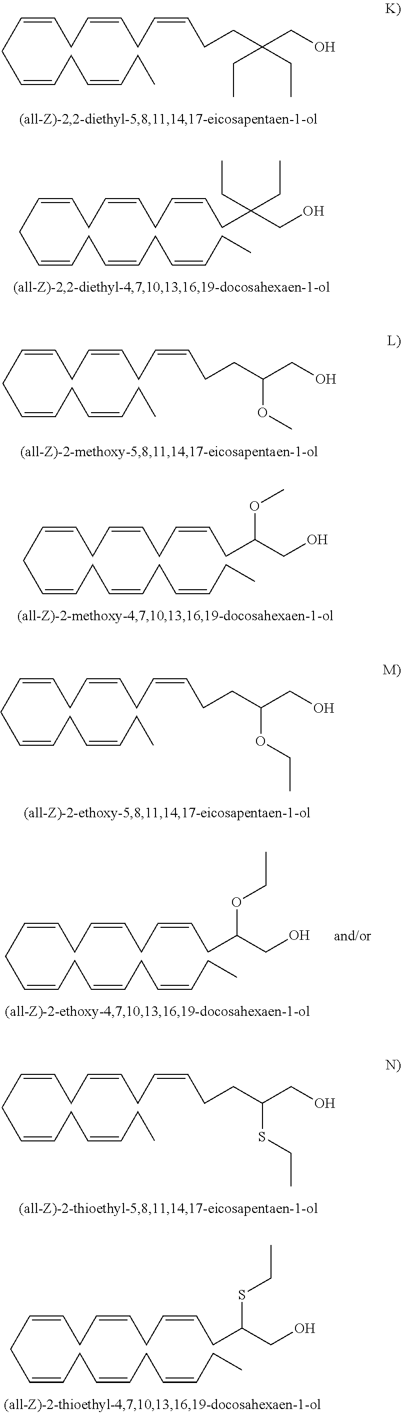

56. A lipid composition according to claim 1, wherein R₁ and R₂ are different.

57. A lipid composition according to claim 56 wherein one or both of the compounds of formulas (I) and (II) are in racemic form.

58. A lipid composition according to claim 56 wherein the compounds of formulas (I) and (II) are in the form of its R stereoisomer or at least one is the R stereoisomer and the other is the S stereoisomer.

59. A lipid composition according to claim 55 wherein the compounds of formulas (I) and (II) are in the form of its S stereoisomer.

60. A pharmaceutical composition comprising a lipid composition according to claim 1.

61. A lipid or pharmaceutical composition according to claim 1 for use in therapy.

62. A pharmaceutical composition according to claim 60, further comprising a pharmaceutically acceptable carrier.

63. A pharmaceutical composition according to claim 59, formulated for oral administration.

64. A pharmaceutical composition according to claim 63, in the form of a capsule, a sachet or in solid dosage form.

65. A pharmaceutical composition according to claim 60, formulated to provide a daily dosage of 1 mg to 10 g of said composition.

66. A pharmaceutical composition according to claim 65, formulated to provide a daily dosage of 1 mg to 1 g of said composition.

67. A pharmaceutical composition according to claim 66, formulated to provide a daily dosage of 50 mg to 200 mg of said composition.

68. A lipid or pharmaceutical composition according to claim 60, further comprising a pharmaceutically acceptable antioxidant.

69. A lipid composition according to claim 68, wherein said antioxidant is tocopherol or an astaxanthin.

70-85. (canceled)

86. A method for the treatment and/or prevention of a condition related to elevated functions of at least one of the human peroxisome proliferator-activated receptor (PPAR) isoforms, comprising administering to a mammal in need thereof a pharmaceutically active amount of a compound according to claim 1.

87. A method according to claim 86, wherein said peroxisome proliferator-activated receptor (PPAR) is PPARα.

88. A method according to claim 86, wherein said peroxisome proliferator-activated receptor (PPAR) is PPARα and/or γ.

89. A peroxisome proliferator-activated receptor (PPAR) α and/or γ agonist, comprising the lipid composition according to claim 1.

90. A method for the treatment and/or the prevention of peripheral insulin resistance and/or a diabetic condition comprising administering to a mammal in need thereof a pharmaceutically active amount of a lipid composition according to claim 1.

91. A method for reduction of plasma insulin, blood glucose and/or serum triglycerides comprising administering to a mammal in need thereof a pharmaceutically active amount of a lipid composition according to claim 1.

92. A method for the treatment and/or the prevention of type 2 diabetes comprising administering to a mammal in need thereof a pharmaceutically active amount of a lipid composition according to claim 1.

93. A method for the prevention and/or treatment of elevated triglyceride levels, non-HDL (LDL and/or VLDL cholesterol levels) comprising administering to a mammal in need thereof a pharmaceutically active amount of a lipid composition according to claim 1.

94. A method for the prevention and/or treatment of a hyperlipidemic condition comprising administering to a mammal in need thereof a pharmaceutically active amount of a lipid composition according to claim 1.

95. A method according to claim 94, wherein said hyperlipidemic condition is hypertriglyceridemia (HTG).

96. A method for increasing serum HDL levels in humans comprising administering to a mammal in need thereof a pharmaceutically active amount of a lipid composition according to claim 1.

97. A method for the treatment and/or the prevention of obesity or an overweight condition comprising administering to a mammal in need thereof a pharmaceutically active amount of a lipid composition according to claim 1.

98. A method for reduction of body weight and/or for preventing body weight gain comprising administering to a mammal in need thereof a pharmaceutically active amount of a lipid composition according to claim 1.

99. A method for the treatment and/or the prevention of a fatty liver disease comprising administering to a mammal in need thereof a pharmaceutically active amount of a lipid composition according to claim 1.

100. A method according to claim 99, wherein said fatty liver disease is non-alcoholic fatty liver disease (NAFLD).

101. A method for treatment of insulin resistance, hyperlipidemia and/or obesity or an overweight condition comprising administering to a mammal in need thereof a pharmaceutically active amount of a lipid composition according to claim 1.

102. A method for the treatment and/or the prevention of an inflammatory disease or condition comprising administering to a mammal in need thereof a pharmaceutically active amount of a lipid composition according to claim 1.

103. A method for the manufacture of a lipid composition according to any claim 1.

104. A method for the manufacture of a lipid composition comprising at least alpha substituted compounds according to claim 1, substantially as described and exemplified in the present specification.

105. A method for the manufacture of a lipid composition according to claim 103, wherein said lipid composition is prepared from a vegetable, a microbial and/or an animal source.

106. A process for manufacture of a lipid composition according to claim 103, wherein said lipid composition is prepared from a marine oil.

107. A process for manufacture of a lipid composition according to claim 106, wherein said lipid composition is prepared from a fish oil or a krill oil.
